(19) United States
(12) Patent Application Publication
(10) Pub. No.: US 2014/0335179 A1
(43) Pub. Date: Nov. 13, 2014

(54) MICRONIZED CRYSTALS OF ATORVASTATIN HEMICALCIUM

(71) Applicants: Rajnish Kumar, Echt (NL); Neeraj Tewari, Echt (NL)

(72) Inventors: Rajnish Kumar, Echt (NL); Neeraj Tewari, Echt (NL)

(73) Assignee: DSM SINOCHEM PHARMACEUTICALS NETHERLANDS B.V., Delft (NL)

(21) Appl. No.: 14/130,606

(22) PCT Filed: Jun. 28, 2012
(86) PCT No.: PCT/EP2012/062592

§ 371 Date: Jun. 2, 2014

(30) Foreign Application Priority Data
Jul. 1, 2011 (EP) 11172440.7

(51) Int. Cl. C07D 207/34; B02C 19/06; A61K 9/28

(52) U.S. Cl. CPC C07D 207/34; A61K 9/28; B02C 19/06
USPC 424/474; 548/537; 514/423; 428/402; 241/18

(57) ABSTRACT

The present invention relates to micronized crystals of atorvastatin hemi-calcium, a method for the preparation of micronized crystals of atorvastatin hemi-calcium and a pharmaceutical dosage form comprising said micronized crystals of atorvastatin hemi-calcium.

### FIELD OF THE INVENTION

The present invention relates to micronized crystals of atorvastatin hemi-calcium, a method for the preparation of micronized crystals of atorvastatin hemi-calcium and a pharmaceutical dosage form comprising said micronized crystals of atorvastatin hemi-calcium.

### BACKGROUND OF THE INVENTION

Atorvastatin ([R-(R*,R*)]-2-(4-fluorophenyl)-β,δ-dihydroxy-5-(1-methylethyl)-3-phenyl-4-[(phenylamino)carbonyl]-1H-pyrrole-1-heptanoic acid hemi calcium salt is a pharmaceutical ingredient useful as an inhibitor of the enzyme 3-hydroxy-3-methylglutaryl-coenzyme A reductase (HMG-CoA reductase) and thus useful as a hypolipidemic and hypocholesterolemic agent.

Atorvastatin, formulations of atorvastatin, processes and key intermediates have been widely disclosed (see e.g. U.S. Pat. No. 4,681,893, U.S. Pat. No. 5,003,080, U.S. Pat. No. 5,097,045, U.S. Pat. No. 5,103,024, U.S. Pat. No. 5,124,482, U.S. Pat. No. 5,149,837, U.S. Pat. No. 5,155,251, U.S. Pat. No. 5,216,174, U.S. Pat. No. 5,245,047, U.S. Pat. No. 5,248,793, U.S. Pat. No. 5,298,627, U.S. Pat. No. 5,273,995, U.S. Pat. No. 5,280,126, U.S. Pat. No. 5,342,952, U.S. Pat. No. 5,397,792, U.S. Pat. No. 5,446,054, U.S. Pat. No. 5,470,981, U.S. Pat. No. 5,489,690, U.S. Pat. No. 5,489,691, U.S. Pat. No. 5,510,488, U.S. Pat. No. 5,686,104, U.S. Pat. No. 5,998,633, U.S. Pat. No. 6,087,511, U.S. Pat. No. 6,126,971, U.S. Pat. No. 6,433,213, U.S. Pat. No. 6,476,235 and documents cited therein). Atorvastatin can exist in crystalline, liquid crystalline, non-crystalline and amorphous forms. Crystalline forms of atorvastatin hemi-calcium are disclosed in U.S. Pat. No. 5,969,156, U.S. Pat. No. 6,121,461 and U.S. Pat. No. 6,605,729.

It has been disclosed that the amorphous forms in a number of drugs exhibit different dissolution characteristics and in some cases different bioavailability patterns compared to the crystalline form (Konno, T., Chem. Pharm. Bull., 1990, 38, 2003-2007). For some therapeutic indications one bioavailability pattern and/or dissolution profile may be favored over another. Variations in dissolution rates can make it advantageous to produce atorvastatin formulations in either crystalline or amorphous forms, depending on the purpose. For example, for some potential uses of atorvastatin (e.g. acute treatment of patients having strokes as described in Takemoto, M.; Node, K.; Nakagami, H.; Liao, Y.; Grimm, M.; Takemoto, Y.; Kitakaze, M.; Liao, J. K., Journal of Clinical Investigation, 2001; 108(10), 1429-1437) a rapid onset of activity may be highly beneficial in improving the efficacy of the drug.

A major drawback of the amorphous form of atorvastatin is that it is known to be less stable compared to crystalline atorvastatin and can undergo degradation during storage under unfavorable conditions. Hence, it would be highly desirable to combine the advantages of the one morphology (i.e. amorphous) with those of the other (i.e. crystalline) and thus there is a need to provide atorvastatin compositions that have the dissolution characteristics of amorphous atorvastatin combined with the favorable stability of crystalline atorvastatin. In view of lengthy regulatory procedures there also is a need to provide such compositions without having to adapt the existing registration dossiers.

Therefore, it is an object of the present invention to provide a stable dosage form of atorvastatin having good stability and bioavailability. It is a further object of the present invention to provide a composition of atorvastatin displaying the characteristics suitable for use in said dosage form.

### DETAILED DESCRIPTION OF THE INVENTION

In a first aspect of the invention there is disclosed crystalline atorvastatin having a well-defined particle size distribution (PSD). In one embodiment the atorvastatin crystals have a PSD wherein d10 is from 2 to 4 μm, d50 is from 5 to 8 μm and d90 is from 10 to 13 μm. More preferably d10 is from 2.5 to 3.5 μm, d50 is from 5.5 to 6.5 μm and d90 is from 11.5 to 12.5 μm.

In the context of the present invention “atorvastatin” refers to the hemi-calcium salt of atorvastatin.

In the context of the present invention “d10” refers to the equivalent diameter where 10% (w/w) of the particles has a smaller diameter, “d50” refers to the equivalent diameter where 50% (w/w) of the particles has a larger diameter, and the other 50% (w/w) has a smaller diameter and “d90” refers to the equivalent diameter where 10% (w/w) of the particles has a larger diameter.

A suitable PSD is one wherein d10 is 3±0.5 μm, d50 is 6±0.5 μm and d90 is 12±0.5 μm as it was surprisingly found that crystals having said PSD can be used instead of amorphous atorvastatin as currently applied in known amorphous atorvastatin based formulations without loss of bioavailability while at the same time displaying enhanced stability.

Surface area and intrinsic dissolution rate (IDR) of amorphous atorvastatin hemi-calcium and the crystalline atorvastatin hemi-calcium having a well-defined PSD of the present invention indicate that the surface area of the latter is significantly higher than that of amorphous samples. Surprisingly, although IDR values for the crystalline atorvastatin hemi-calcium having a well-defined PSD of the present invention are lower (mean value of 0.132 mg/cm²/min with variations between 0.109 mg/cm²/min to 0.281 mg/cm²/min) than those of amorphous atorvastatin (mean value of 0.281 mg/cm²/min with variations between 0.183 mg/cm²/min to 0.320 mg/cm²/min), the bioavailability still is adequate. Hence preferred IDR values for the crystalline atorvastatin hemi-calcium having a well-defined PSD of the present invention are from 0.1 mg/cm²/min to 0.3 mg/cm²/min, more preferred from 0.12 mg/cm²/min to 0.28 mg/cm²/min, most preferably from 0.13 mg/cm²/min to 0.26 mg/cm²/min.

In a second aspect of the invention there is disclosed a method for the preparation of crystalline atorvastatin having a well-defined PSD. Crystalline atorvastatin can readily be prepared as described in U.S. Pat. No. 4,681,893, U.S. Pat. No. 5,273,995 U.S. Pat. No. 5,969,156 and other documents. Following preparation of crystalline atorvastatin, the resulting product is micronized. Micronized forms of atorvastatin are described in EP 1808162, EP 1923057 and WO 2009/140341. However these documents do not disclose precise micronization conditions required to obtain micronized crystalline atorvastatin particles that are suitable for uses in applications and formulations that hitherto where used for amorphous atorvastatin since these documents are not concerned with this particular issue (i.e. EP 1808162 and EP 1923057 deals with the reduction of the food effect encountered by administration of atorvastatin whereas WO 2009/140341 is concerned with the development of formulations of atorvastatin displaying similar characteristics as known formulations of crystalline atorvastatin. In order to achieve micronized crystalline atorvastatin particles that are suitable for uses in amorphous atorvastatin applications, a well-defined PSD is needed including not only well-defined d50 and d90 values but also a well-defined d10 value. Such particles are achievable by the micronization process of the present invention.

In the context of the present invention micronization may be a mechanical process that involves the application of force to a particle, thereby resulting in breaking of the particle. Such force may be applied by collision of particles at high speeds. Micronization may be carried out by grinding, using an air-jet micronizer, ball mill or pin mill to produce micronized particles. Preferably said micronization is carried out in a micronizer equipped with a jet nozzle operating at a compressed air pressure of from 5 kg/cm² to 100 kg/cm², preferably of from 6 kg/cm² to 50 kg/cm², more preferably of from 6 kg/cm² to 25 kg/cm².

The size of a particle is determined by any of the methods commonly known in the art such as sieve analysis, sedimentation, microscopy and light (laser) diffraction techniques. Traditionally, sieves are used for determining the PSD. They function well in the size range of 40 μm to several mm. The sieves mostly used for analytical purposes are wire mesh screens. The apertures have square cross sections. All apertures of the same sieve are basically of the same magnitude. Nominal aperture size of the screen is commonly denoted in μm or mm. The aperture size of analytical sieves used to be characterized by a mesh number which is related to the number of wires per surface area in the weave. Thus actual aperture size depends on the thickness of the wire. As a consequence, a high mesh number corresponds to a small aperture size, and vice versa. In different countries different systems have been in use, e.g. ASTM (USA), BS (UK), Tyler (UK) and DIN (Germany). Often, sieve analyses and PSD specifications made up in one of these denotations are still encountered. For accurate determinations, sieves should be calibrated. This can be done microscopically or by using standard reference materials of an exactly known PSD.

Light (laser) diffraction techniques function by dispersion of particles in a medium, such as air or in a liquid, followed by scattering of the light beams by which they are hit. The light is not scattered equally in all directions and some directions are preferred over others and consequently a light scattering pattern emerges. This pattern is strongly related to the size and the size distribution of the particles and theories are available that quantitatively relate the scattering pattern to the PSD. Laser diffraction apparatus use the scattering behavior of light by dispersed particles and comprises a laser (providing a narrow, monochromatic light beam), a system of lenses (to focus the laser beam on the sample and to focus the scattered light on the detectors), a sample cell (in which the sample is contained in a dispersed state), a set of light detectors (to detect and measure the intensities of the scattered light) and a computerized algorithm (to convert the measured pattern of intensities of the scattered light into a PSD). The PSD of the sample is constructed by putting the measured light intensities into the theoretical equations of either the Fraunhofer theory or the Mie theory and performing the calculations by computer. Various instruments are available commercially. All make use of the same principle, although differences exist between instruments of different producers. Differences may include the optical system (lenses), the number of detectors, the dispersion medium (air or liquid), the scattering model applied (Fraunhofer or Mie) and the software. Widely used laser diffraction instruments include those from Malvern, Sympatec and Beckman/Coulter. The results of sampling with instruments of different brands on the same sample will not necessarily be the same. Differences in results can have both theoretical and experimental causes. Important causes of the differences in resulting PSD are the dispersion medium, the number of detectors and the scattering model used. The preferred method for the present invention is measurement using a Malvern apparatus.

In a third aspect of the invention there is disclosed a pharmaceutical dosage form comprising atorvastatin of the first aspect of the invention. The dosage form of the invention may contain any pharmaceutically acceptable excipient known in the art. In one embodiment, the excipient is at least one of vitamin E, hydroxypropylcellulose, microcrystalline cellulose, crospovidone, sodium bicarbonate, mannitol, meglumine, polacrilin including polacrilin potassium, polyvinylpyrrolidone, calcium phosphate such as dibasic calcium phosphate anhydrous, lactose including the monohydrate, colloidal silicone dioxide, talc, magnesium stearate, croscarmellose, sodium carbonate, polyplasdone, magnesium aluminum silicate, sodium stearyl fumarate, or a coating such as Opadry. In another embodiment, the excipient is at least one of calcium oxide, magnesium oxide, calcium magnesium carbonate, carbonates or bicarbonates of sodium, potassium or ammonium; ammonium or alkali metal salts of phosphoric acid or pyrophosphate; ammonium or alkali metal salts of carboxylic acids or fatty acids; calcium magnesium acetate, ammonium or alkali metal salts of aspartic or glutamic acid; carbonates of lysine or arginine; bicarbonates of lysine, arginine, cystine or histidine; free base forms of lysine, arginine, tryptophan, histidine, asparagine or glutamine; carboxylic acid salts of lysine, arginine or histidine; salt forms of cystine, phenols, biophenols or flavonoids, vitamin P, tyrosine, isoflavones, polymers carrying amine functions, polymers carrying acid functions in their salt forms, polyvinyl acetate or phthalate; and peptides or proteins with iso-electric point greater than 4.5.

The present invention also encompasses methods of preparing the pharmaceutical dosage forms of the invention. In one embodiment, the method of preparing the pharmaceutical dosage form of the invention includes preparing a mixture of atorvastatin and at least one the excipients mentioned above. Dosage forms of the invention may be prepared in accordance with customary processing techniques for pharmaceutical formulations wherein the ingredients are suitably processed and formulated into a dosage form, e.g., compressed into a tablet, with pharmaceutically acceptable excipients. In a preferred embodiment, the method includes formulating the dosage form into an oral dosage form, such as a tablet. One preferred unit dosage form for atorvastatin is a tablet the preparation of which may be as described in U.S. Pat. No. 7,790,197 or Ahjel, S. W.; Lupulease, D., Farmacia, 2009; 57(3), 290-300. For active drugs in tablets to be rapidly absorbed once swallowed, it is generally important for the tablet to disintegrate rapidly once exposed to fluids in the gastrointestinal tract. At the same time, it is important that the tablets be sufficiently hard that they do not fracture or chip during manufacturing, handling or storage. These seemingly contradictory needs can be met by addition of disintegrants to the composition. A number of disintegrants for compositions of atorvastatin have been disclosed in the prior art including calcium carboxymethylcellulose, starch and croscarmellose sodium (see U.S. Pat. No. 5,686,014 and U.S. Pat. No. 6,126,971). Preferably, the tablet is coated with a coating.

In one embodiment, the method of preparing the pharmaceutical dosage form of the invention includes preparing a mixture of atorvastatin and a pharmaceutically acceptable excipient; granulating the mixture to form granules; and formulating the granules into the dosage form. The method of preparing the pharmaceutical dosage form of the invention includes preparing a mixture of atorvastatin and at least one pharmaceutically acceptable excipient; preparing a solution comprising vitamin E and hydroxypropylcellulose; granulating the solution with the mixture to obtain granules; combining at least one of crospovidone or colloidal silicone dioxide with the granules and adding at least one of magnesium stearate or talc to form the dosage form. In a preferred embodiment, at least one of microcrystalline cellulose, crospovidone, sodium bicarbonate, meglumine, polacrilin, calcium phosphate, or lactose is added to the mixture with atorvastatin before granulation. Preferably, all ingredients are added into the mixture before granulation, and more preferably, in the order of atorvastatin hemi-calcium, microcrystalline cellulose, crospovidone, sodium bicarbonate, meglumine, polacrilin, calcium phosphate, and lactose. The granulate mixture so obtained may be compressed into a tablet which is subsequently coated. Tablets may be coated with coatings comprising phthalic acid cellulose acetate, hydroxypropylmethyl-cellulose phthalate, polyvinyl alcohol phthalate, carboxymethylethylcellulose, a copolymer of styrene and maleic acid, a copolymer of methacrylic acid and methyl methacrylate and the like and may be employed with suitable plasticizers and/or extending agents. A coated tablet may have a coating on the surface of the tablet or may be a tablet comprising a powder or granules with an enteric-coating.

In a preferred embodiment, atorvastatin is present in the tablet in an amount of from about 5% to about 20%, more preferably from about 5% to about 10%, and more preferably in an amount of about 8%. Preferred unit dosages of the pharmaceutical compositions of this invention typically contain from 0.5 to 100 mg of atorvastatin. More usually, the atorvastatin is present in a unit dosage in an amount of from 2.5 mg to 80 mg. Dosage forms may include diluents, such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents like calcium carbonate and calcium diphosphate and other diluents known to the pharmaceutical industry. Yet other suitable diluents include waxes, sugars and sugar alcohols like mannitol and sorbitol, acrylate polymers and copolymers, as well as pectin, dextrin and gelatin.

### LEGEND TO THE FIGURES

FIGS. 1-3 are the X-ray powder diffraction patterns recorded from three different samples of crystalline atorvastatin having a well-defined PSD. X-axis: 2θ value (deg). Y-axis: intensity (cps). The following distinct peaks can be discerned at 2θ values of: 9.1±0.3, 9.4±0.3, 10.2±0.3, 11.8±0.3, 12.1±0.3, 16.9±0.3, 17.0±0.3, 17.2±0.3, 19.4±0.3, 21.3±0.3, 21.5±0.3, 21.9±0.3, 22.6±0.3, 23.2±0.3 and 23.6±0.3.

FIGS. 4-6 are the infrared spectra recorded from three different samples of crystalline atorvastatin having a well-defined PSD. X-axis: wavelength (cm⁻¹). Y-axis: transmission (% Ts). FIG. 4 concerns the same sample as that of FIG. 1, FIG. 5 concerns the same sample as that of FIG. 2 and FIG. 6 concerns the same sample as that of FIG. 3.

### METHODS

### Measurement of Particle Size Distribution (PSD)

Instrument: The PSD was determined using a Malvern Mastersizer 2000 equipped with hydro 2000S(A), flow cell, Fourier lenses and a multi-element detector
Data handling system: Malvern Mastersizer 2000 version 5.21
Technique used: Wet method
Optical parameters: Optical presentation comprising of:
   Particle RI: 1.5
   Absorption: 0
   Dispersant RI: 1.375 or 1.4
Analysis Model: General purpose
Sample measurement snaps: 5000
Background meas. snaps: 5000
No. of measurement cycles: 5
Delay between measurements: 5 sec
Preparation of sample solution: About 25 mg of the sample was weighed in a beaker and about 20 mL of dispersion medium was added with constant stirring and sonification for 30 sec resulting in a uniform dispersion. After sonification the measurement was performed after the obscure value was stabilized between 10-20%
Procedure:
   a. The flow cell was assembled to the instrument
   b. Dispersion medium poured to Hydro 2000S(A) unit
   c. Stirrer speed was set at 2200 rpm
   d. Instrument was aligned after feeding the required parameters
   e. Background was measured
   f. Sample was analyzed by recording five measurements for each sample preparation and the mean RSD values were collected at D(v, 0.1), D(v, 0.5) and D(v, 0.9)

### Surface Area Determination

Specific surface area of the samples was determined using nitrogen gas sorption (Smartsorb, Smart Instruments, Mumbai, India). Prior to measurements, weighed samples were regenerated by degassing to remove moisture and contamination. The regenerated sample was dipped in liquid nitrogen and the quantity of the adsorbed gas was measured using thermal conductivity detector and then integrated using electronic circuit in terms of counts. The instrument was calibrated by injecting known quantity of nitrogen. The measured parameters were then used to calculate surface area of the sample by employing the adsorption theories of Brunauer, Emmett and Teller (BET).

### Determination of Intrinsic Dissolution Rate (IDR)

IDR of crystalline and amorphous atorvastatin hemi-calcium samples was performed by the stationary disc method using USP 24 apparatus. The discs were prepared by compacting 100 mg of powder at 800 psi pressure in a hydraulic press (Hydraulic Unit Model 3912, Carver Inc., WI) with a dwell time of 15 sec, in 8 mm punch die set (surface area 0.5 cm²). The dissolution studies were performed in 500 ml of 50 mM sodium dihydrogen phosphate buffer (pH 6.8) containing 2% w/v sodium lauryl sulphate maintained at a temperature of 37±0.5° C. and a rotational speed of 100 rpm. Samples were withdrawn at regular intervals, replaced with dissolution medium, and analyzed by UV spectrophotometry at 242 nm up to 60 minutes.

### Example

### Preparation and micronization of atorvastatin hemi-calcium from 2-((4R,6R)-6-(2-(3-(phenylcarbamoyl)-5-(4-fluorophenyl)-2-isopropyl-4-phenyl-1H-pyrrol-1-yl)ethyl)-2,2-dimethyl-1,3-dioxan-4-yl)acetic acid isopropyl ester

2-((4R,6R)-6-(2-(3-(phenylcarbamoyl)-5-(4-fluorophenyl)-2-isopropyl-4-phenyl-1H-pyrrol-1-yl)ethyl)-2,2-dimethyl-1,3-dioxan-4-yl)acetic acid isopropyl ester (75 kg, 117 mol) was added to 1125 L of methanol. The mixture was stirred at 35-37° C. until clarity. The solution was cooled to 25-27° C. Aqueous HCl solution was added (made from 19 kg concentrated HCl and 64 L of water) and the reaction was stirred for 2 h at 25-27° C. Next, the mixture was concentrated under vacuum in 3.5 h at 20-22° C. to ˜50% of its original volume. Then methanol (825 L) was added and the mixture stirred for 45 min. HPLC analysis revealed the starting material to be less than 0.03%. To the mixture aqueous NaOH was added (made from 15 kg NaOH and 560 L water) keeping the temperature below 30° C. to give a pH of 12.2. The solution was stirred for 2 h at 25-27° C. keeping the pH at not less than 12. The reaction mixture was concentrated to about 900 L under vacuum at a temperature of 20-22° C. in 5 h. Next, 750 L of water and 450 L of methyl-t-butyl ether were added and stirred for 15 min. The reaction mixture was allowed to settle and the phases were separated. Again 450 L of methyl-t-butyl ether was added and stirred for 15 min. The reaction mixture was allowed to settle and the phases were separated. After heating the aqueous layer to 35-37° C., 7.5 kg active carbon was added followed by stirring for 30 min. The reaction mixture was filtered through a Hyflo bed and the carbon/hyflo bed washed with water/methanol (135 L water/15 L methanol). The resulting solution was concentrated under vacuum, followed by addition of 150 L of water and 37.5 L of methyl-t-butyl ether. The temperature was increased to 45-47° C. and the pH adjusted to 8.6-8.8 with aqueous acetic acid (3 L acetic acid in 60 L of water). After heating the reaction mixture until 47-50° C., 7.5 kg of Atorvastatin hemi-calcium Polymorph I seed was added, followed by addition in 1 h of a solution of 14.5 kg Ca-acetate in 375 L of water. The mixture was heated to 55-58° C. and maintained at this temperature for 30 min. The slurry was then cooled to 40-45° C. and stirred for 3 h. The solid was isolated by centrifugation and the obtained wet-cake re-slurried in 1125 L of water at 40-45° C. After stirring for 1 h, the solid was isolated by centrifugation and dried under vacuum at 50-55° C. Weight 63.5 kg.

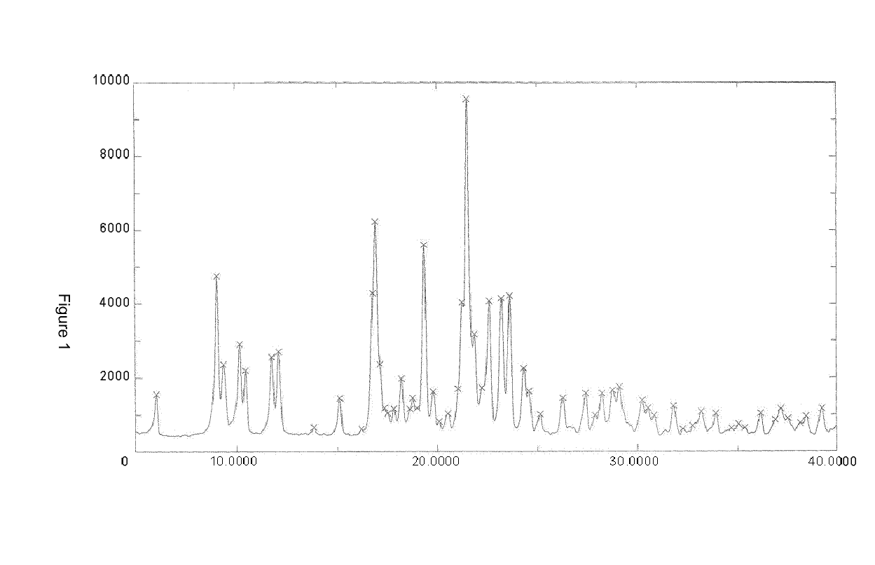

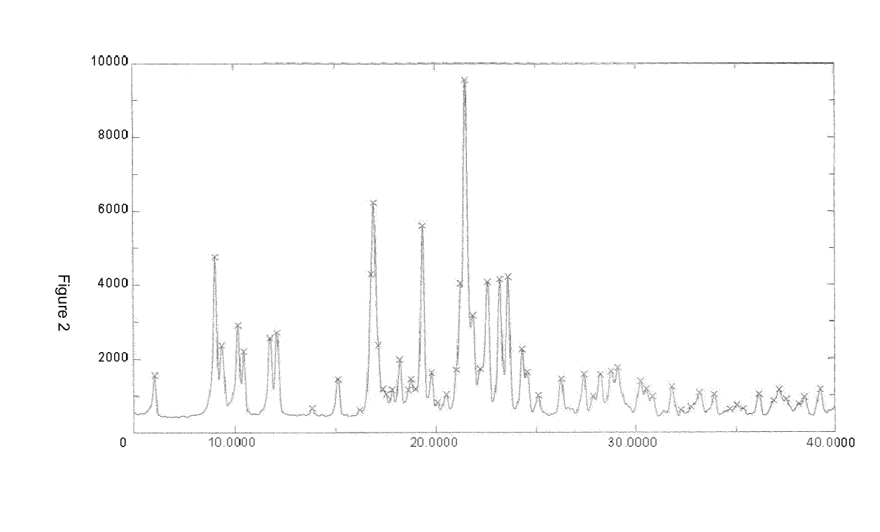

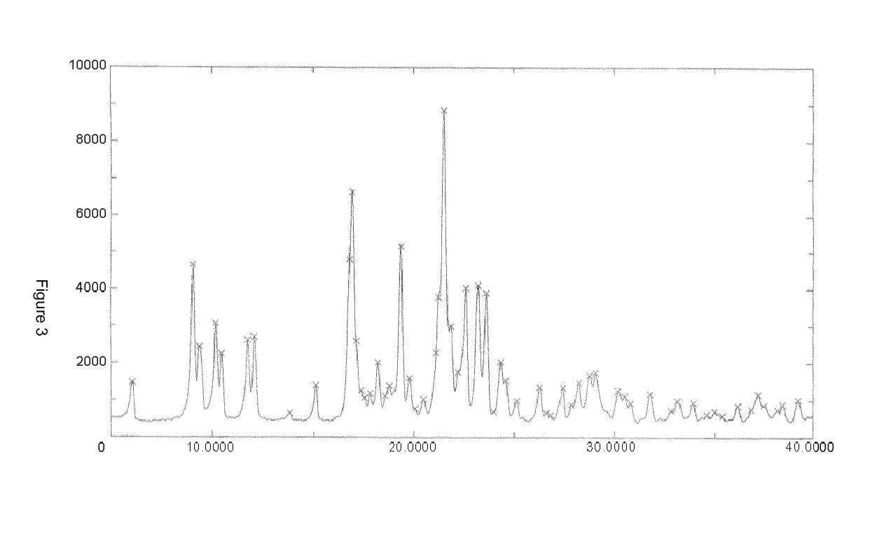

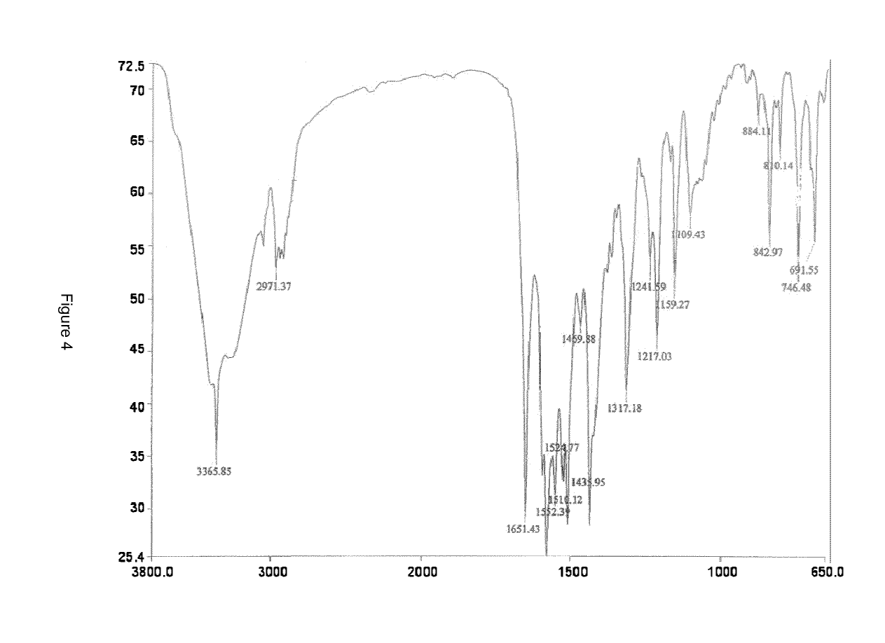

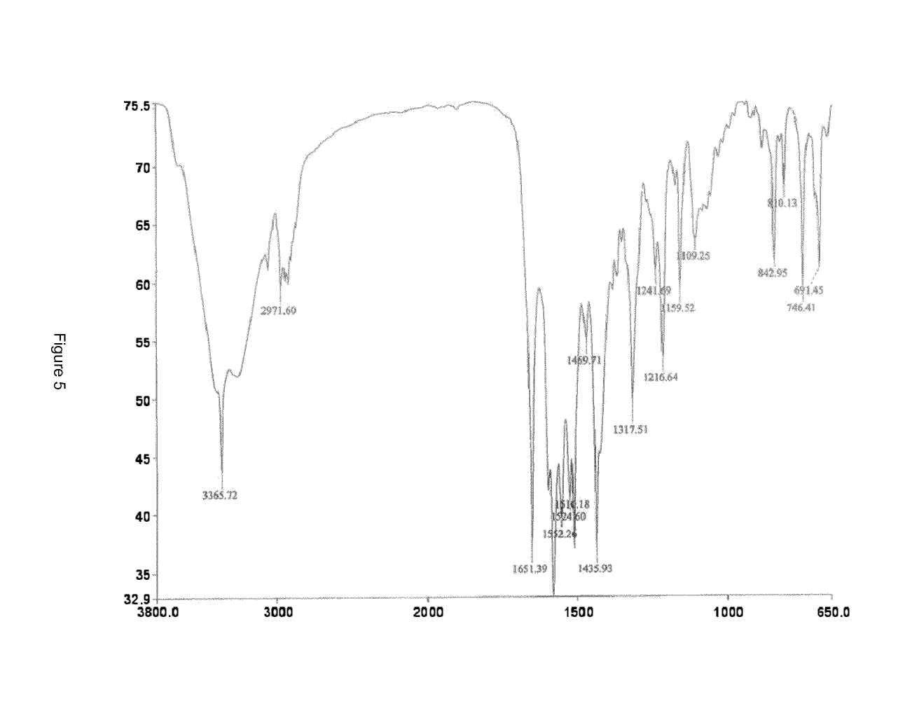

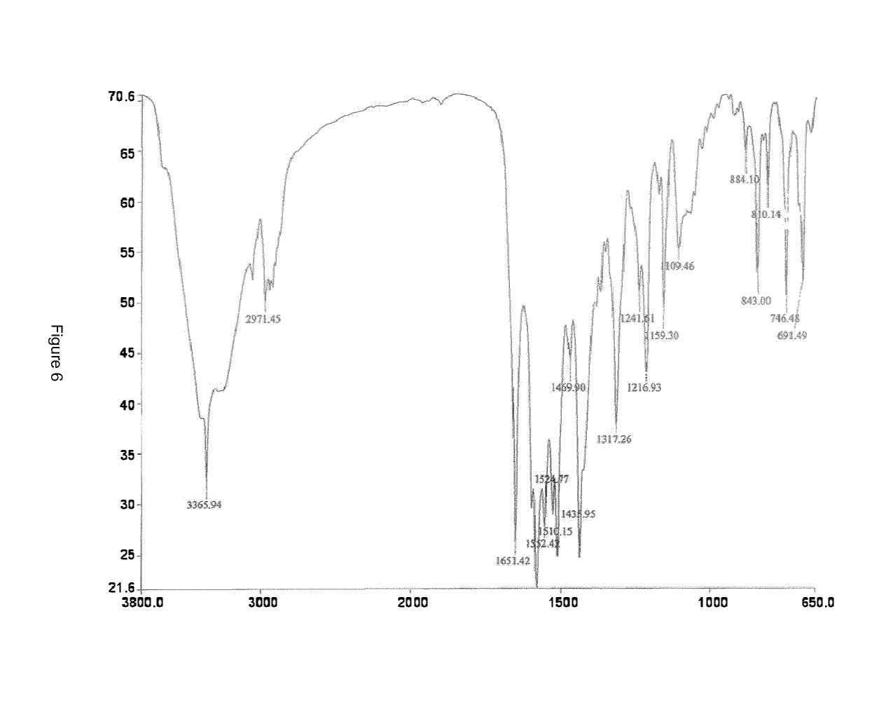

The crystals so obtained were micronized using a micronizer. In this process air was supplied through a jet nozzle non-micronized product to be ground was conveyed from a hopper by a screw feeder. The compressed air pressure was no less then 6 kg/cm²). The pressure at the nozzle was 4-5 kg/cm²). The obtained PSD was d10=3 μm, d50=6 μm, and d90=12 μm (mean value of 35 separate batches). The surface areas of three different samples so obtained were 8.550±0.051 m²/g, 8.384±0.303 m²/g, and 8.684±0.216 m²/g (in comparison, amorphous samples displayed surface areas between 2.844±0.094 m²/g and 4.748±0.081 m²/g). The IDR values of the same three samples were measured to be 0.146±0.013 mg/cm²/min, 0.130±0.017 mg/cm²/min and 0.109±0.006 mg/cm²/min, respectively. Infrared and XRD spectra of the three samples are as outlined in FIGS. 1-6.

1. Crystalline atorvastatin hemi-calcium having a particle size distribution wherein d10 is from 2 to 4 m, d50 is from 5 to 8 m and d90 is from 10 to 13 m.

2. Crystalline atorvastatin hemi-calcium according to claim 1 having a particle size distribution of d10=3±0.5 m, d50=6±0.5 m and d90=12±0.5 m.

3. Crystalline atorvastatin hemi-calcium according to claim 1 having an XRD spectrum with peaks at 2θ values of 9.1±0.3, 16.9±0.3, 17.0±0.3, 19.4±0.3, 21.3±0.3, 21.5±0.3, 22.6±0.3, 23.2±0.3 and 23.6±0.3.

4. Crystalline atorvastatin hemi-calcium according to claim 3 further comprising peaks at 2θ values of 9.4±0.3, 10.2±0.3, 11.8±0.3, 12.1±0.3, 17.2±0.3, and 21.9±0.3.

5. Crystalline atorvastatin hemi-calcium according to claim 3 wherein the intensity of any of said peaks is more than 10% of the intensity of the most intense of said peaks.

6. Crystalline atorvastatin hemi-calcium according to claim 1 having an intrinsic dissolution rate of from 0.12 mg/cm²/min to 0.28 mg/cm²/min.

7. Method for the production of crystalline atorvastatin hemi-calcium according to claim 1 comprising micronizing crystalline atorvastatin hemi-calcium.

8. Method according to claim 7 wherein said micronization is carried out in a micronizer equipped with a jet nozzle operating at a compressed air pressure of from 6 kg/cm² to 50 kg/cm².

9. A pharmaceutical dosage form comprising crystalline atorvastatin according to claim 1.

10. A pharmaceutical dosage form according to claim 9 which is a coated tablet.
